(19) United States
(12) Patent Application Publication
(10) Pub. No.: US 2010/0069905 A1
(43) Pub. Date: Mar. 18, 2010

(54) MINIMALLY INVASIVE OSTEOSYNTHESIS ASSEMBLY

(76) Inventor: Oren Gavriely, Haifa (IL)

Correspondence Address: YORAM TSIVION, P.O. BOX 1307, PARDES HANNA, 37111, IL

(21) Appl. No.: 12/302,951

(22) PCT Filed: Jun. 1, 2007
(86) PCT No.: PCT/IB07/52068

§ 371 Date: Dec. 1, 2008

Related U.S. Application Data
(60) Provisional application No. 60/809,818, filed on Jun. 1, 2006.

(51) Int. Cl. A61F 5/04; B23P 11/00

(52) U.S. Cl. 606/59; 29/525.01

(57) ABSTRACT

A minimally-invasive osteosynthesis assembly includes bone-screwable members, interlocking bridging members, and locking elements. The bone-screwable members are screwed into the tissue a bone fragment constituting an anchoring point to the bone fragment and the interlocking bridging members are applied to different bone-screwable members so as when the locking elements are tighten the bone-screwable members and the interlocking bridging members are frictionally engaged to each other and form rigid structural framework implemented for osteosynthesis.

### CROSS-REFERENCE TO RELATED APPLICATIONS

The present application claims the benefit of priority to U.S. Provisional Patent Application Ser. No. 60/809,818, filed Jun. 1, 2006, entitled “Minimally invasive internal fixation;” the aforementioned application is incorporated herein by reference.

### FIELD OF THE INVENTION

The present invention relates to minimally-invasive medical devices and procedures in general. More specifically, the present invention relates to an external support assembly for orthopedic fixation of bone fractures and a related method.

### BACKGROUND OF THE INVENTION

Break of the mechanical integrity of bones may result from trauma or as part of surgical orthopedic reconstruction procedures.

To achieve healing, the bone fragments and elements must be aligned properly and then firmly stabilized for rather prolonged a period of time. One of the common methods of bone stabilization is by internal fixation whereby special hardware is implanted adjacent to the bone elements to bridge the fracture line. Typically this hardware consists of strong perforated metal plates that are affixed to the bone by screws or rods. The application of the plate is done after mechanical means are used to position the bone elements in place.

The usual process begins after anesthesia, disinfection and preparations are accomplished. An incision is made in the skin, the soft tissues are dissected and divided and the bone surface is exposed. Repositioning of the bone elements and fragments is performed, either manually, or with tools and temporary stabilization is often done using pins. A plate is then selected and held to the bone surface. Holes are drilled through the plate's perforations into the bone and screws are used to tighten the plate to the bone surface.

Once the plate is secured the soft tissues such as muscles, fascia, and ligament are repaired and the skin is closed. While this is the prevailing method, it has several recognized drawbacks: it requires a long and disfiguring skin incision; it requires extensive soft tissue dissections; bone fragments manipulation is often difficult; screw positions are rigidly determined by the shape and perforations in the plate; plates are in mechanically suboptimal disadvantage due to uneven distribution of forces.

Several considerable drawbacks inherent to the presently prevailing orthopedic practice of bone fragments fixation are overcome by the novel minimally-invasive osteosynthesis assembly that is potentially much less invasive, requiring few small incisions and less soft tissue dissection, and furthermore provides a means for manipulating bone fragments.

### SUMMARY OF THE INVENTION

There are provided in accordance with the present invention a set of components of minimally-invasive external support assembly for orthopedic fixation of bone fractures. There is further provided a process of assembling the aforementioned components together into a firm structural framework and furthermore a method of implementing the method for osteosynthesis of fractured bone, whereby a mechanical support of the bone is achieved. In addition, accessory tools needed to implement the method provided by the invention are disclosed.

### BRIEF DESCRIPTION OF THE DRAWINGS

The present invention will be understood and appreciated more fully from the following detailed description taken in conjunction with the appended drawings in which:

FIG. 1. is an isometric representation of an exemplary bone-screwable member;

FIG. 2. is an isometric representation of an exemplary interlocking bridging member, having straight bridging element;

FIG. 3. is an isometric representation of another exemplary interlocking bridging member having straight bridging element and a relatively smaller interlocking element;

FIG. 4. is an isometric representation of yet another exemplary interlocking bridging member having straight bridging element and toroidal interlocking element;

FIG. 5. is an isometric representation of an exemplary interlocking bridging member having arc-like bridging element;

FIG. 6. is an isometric representation of another exemplary bone-screwable member having arc-like bridging element and a relatively small interlocking element;

FIG. 7. is an isometric representation of an exemplary assembly of bone-screwable member and two interlocking bridging members;

FIG. 8. is an isometric representation of an exemplary locking element;

FIG. 9. is an isometric representation illustrating an anchoring point on tubular bone fragment;

FIG. 10. is a schematic representation of osteosynthesis assembly implemented for fixation of a fractured bone, in accordance with some preferred embodiment of the present invention.

While the invention is susceptible to various modifications and alternative forms, specific embodiments thereof have been shown by way of example in the drawings. The components in the drawings are not necessarily to scale, emphasis instead being placed upon clearly illustrating the principles of the present invention. It should be understood, however, that the description herein of specific embodiments is not intended to limit the invention to the particular forms disclosed, but on the contrary, the intention is to cover all modifications, equivalents, and alternatives falling within the spirit and scope of the invention.

### DISCLOSURE OF THE INVENTION

Illustrative embodiments of the invention are described below. In the interest of clarity, not all features Illustrative embodiments of the invention are described below. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which vary from one implementation to another. Moreover, it will be appreciated that such a development effort might be complex and time-consuming, but would nevertheless be a routine undertaking for those of ordinary skill in the art having the benefit of this disclosure.

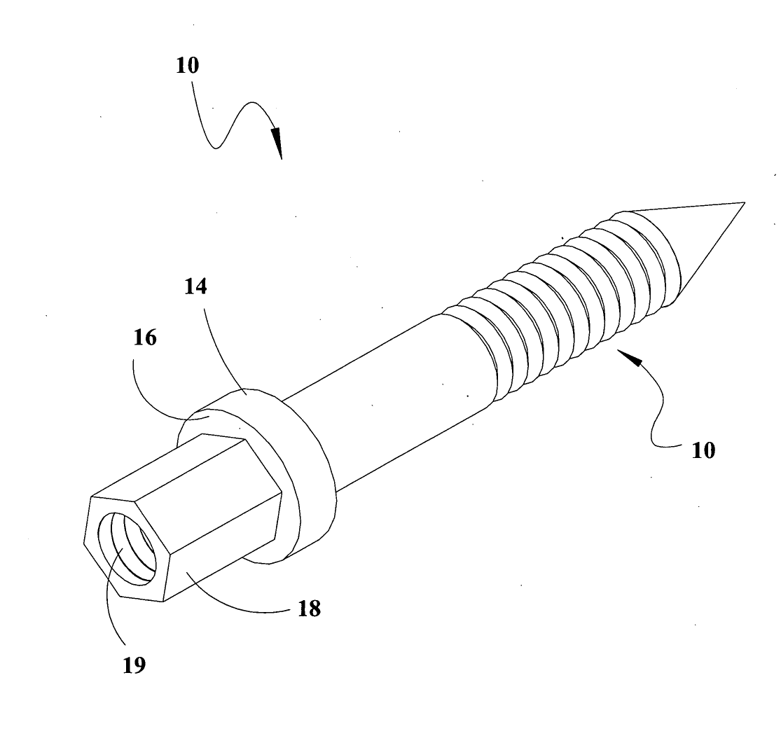

According to the present invention, the minimally-invasive osteosynthesis assembly consists of a plurality of bone-screwable members (BSM), at least one interlocking bridging member (IBM), and a plurality of locking elements (LE) for engaging IBMs to BSMs and or IBMs to other IBMs. The BSM has a bone-screwable portion, IBM engaging portion and LE connecting portion (LECP). Reference is now made to FIG. 1, showing exemplary IBM 10, having bone-screwable portion 12, IBM engaging portion 14 with serrated upper face surface 16, and protruding LECP 18 having cylindrical recess 19 coaxial with the main axis of IBM 10.

The bone-screwable portion is essentially an elongated cylindrical or slightly conical, optionally having a pointed tip; it also typically has a screw thread along at least part of its exterior surface.

In regard to the main axis, IBM engaging portion is typically of an annular shape, and optionally may have a bigger exterior circumference than the exterior circumference of the bone-screwable portion. IBM engaging portion has an upper face and a bottom face. The bottom face faces the bone and may accordingly be used as a spacer preventing the bone-screwable portion from penetrating farther into the bone than predetermined by the contact between the bottom face and bones' outer surface. The IBM's upper face faces the opposite direction, away from the bone, and may be essentially planar or curved. The upper face may further be characterized by having a serrated or rough surface so as to enhance frictional engagement between the IBM and the BSM.

In preferred embodiments, the LECP protrudes from the upper face of the IBM, forming a coaxial extension of the main axis the IBM. The LECP optionally has a screw thread along its surface. However, according to some embodiments the LECP is a cylindrical recess within the IBM underneath IBM's upper face, optionally with a screw thread. In the case of a protruding LECP, it may further have a cylindrical recess, coaxial with the main axis, optionally also with a screw thread.

The IBM engaging portion and or the LECP can further have various grooves, ridges, notches, pawls and or screw threads for complementary instruments that inter alia can be used for applying a rotational torque and or other physical effects to the IBM for rotating and or manipulating it.

The IBM consists of two individual interlocking elements and a bridging element in between. Interlocking elements are versatile and can be of different types.

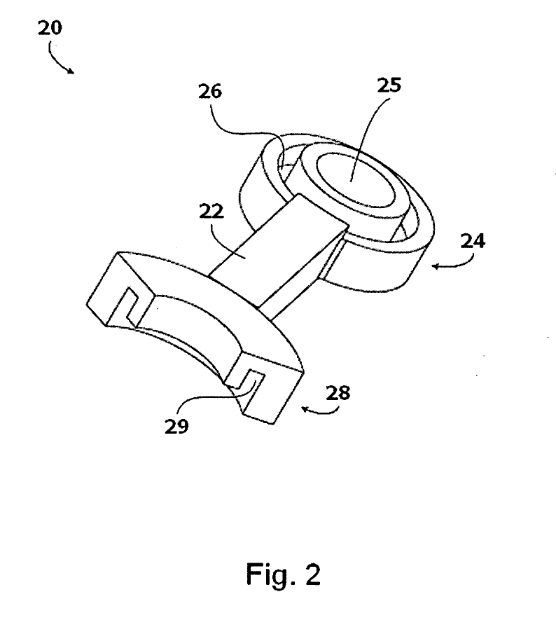

Reference is now made to FIG. 2, showing exemplary IBM 20 having straight bridging elements 22, annular interlocking element 24 with aperture 25 and groove 26, and an annular sector interlocking element 28 with groove 29.

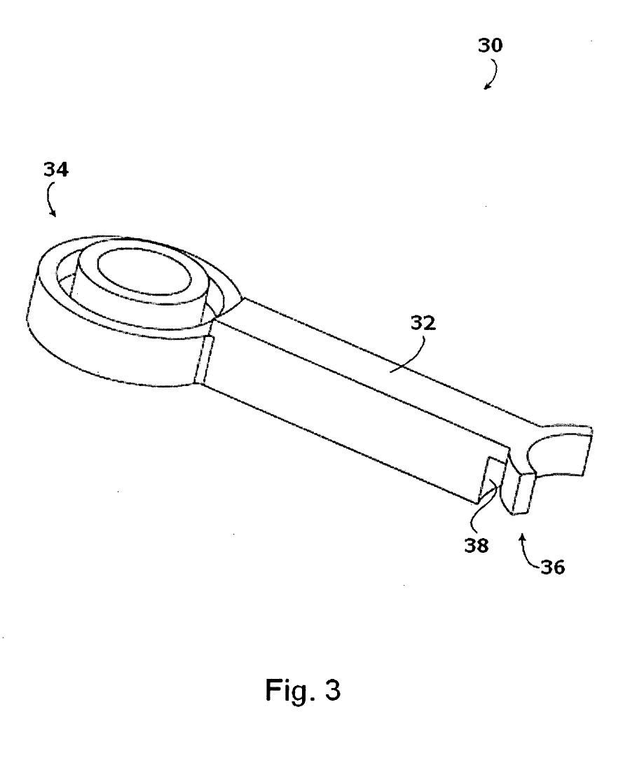

Reference is now made to FIG. 3, showing exemplary IBM 30 having straight bridging elements 32, annular interlocking element 34, and a relatively smaller interlocking element 36 with groove 38.

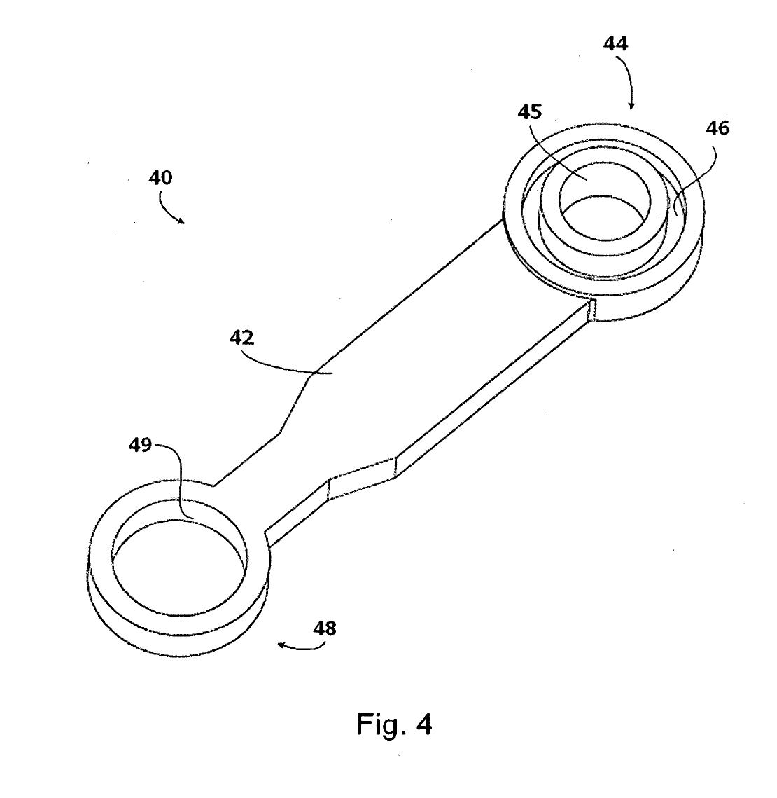

Reference is now made to FIG. 4, showing exemplary IBM 40 having a straight bridging element 42, annular interlocking element 44 with aperture 45 and groove 46, and annular interlocking element 48 with aperture 49.

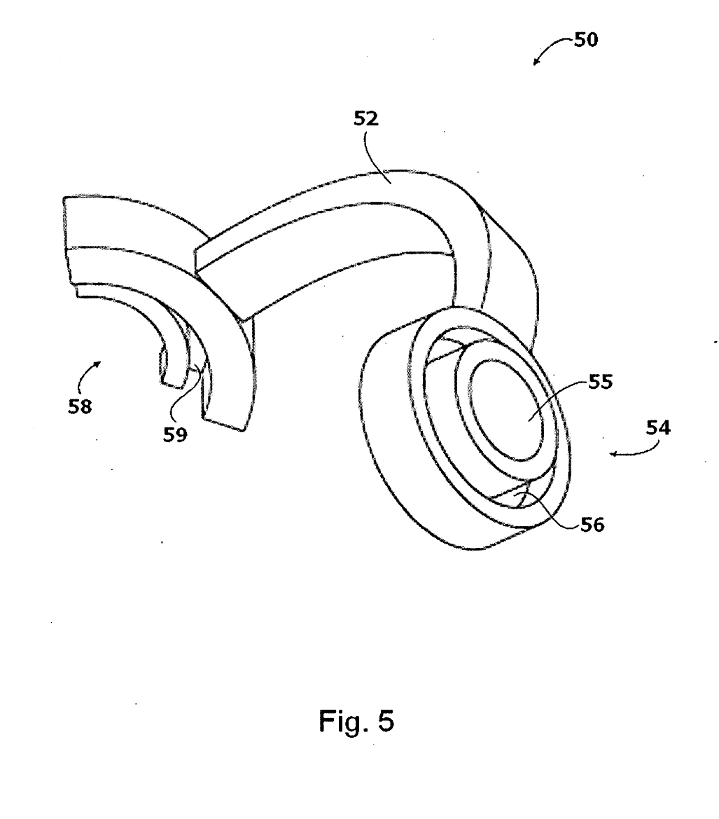

Reference is now made to FIG. 5, showing exemplary IBM 50 having arcuate bridging elements 52, annular interlocking element 54 with aperture 55 and groove 56, and sector of an annulus interlocking element 58 with groove 59.

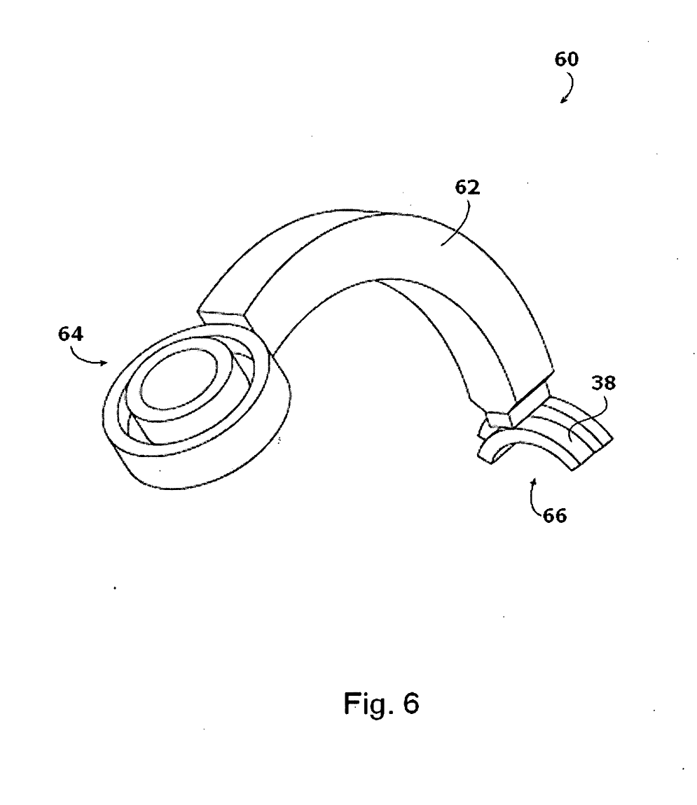

Reference is now made to FIG. 6, showing exemplary IBM 60 having arcuate bridging elements 62, annular interlocking element 64, and a relatively smaller interlocking element 66 with groove 68.

Some interlocking elements have a shape complementary to the IBM engaging portion of BSMs, for anchoring the IBMs to the bone; while some have a shape complimentarily matching other interlocking elements, for tandem assembly of structural framework implemented for osteosynthesis. Interlocking elements can further have a groove and or ridge for achieving physical engagement with other interlocking elements. According to some embodiments, the interlocking elements are essentially annular, having an aperture in the centre, and can be threaded on a matching protruding LECP; accordingly, the complimentarily matching interlocking elements have a shape of a sector of an annulus.

According to some other embodiments, the interlocking elements essentially have shape of a dome-like shell, to match a curved upper face of IBMs, and a curved face of other dome-like shell interlocking elements.

The faces of the interlocking elements may further have a serrated or ragged surface so as to facilitate an enhanced frictional engagement between the interlocking element and BSM and or other interlocking element.

Bridging elements can be of different types also. Some bridging elements have an essentially straight form conferring a linear relative orientation to the interlocking elements at the ends thereof; while some bridging elements are structured, such as arcuate, conferring an angular relative orientation to the interlocking elements at the ends thereof. Various other forms and shapes are possible and in accordance with the present invention.

According to some embodiments bridging elements constitute a monolithic structure with the interlocking elements. However, according to some more elaborate embodiments interlocking elements may consist of several constituents among which are: telescopic constituents for adjustably conferring to the bridging elements a desired lengths, and fixable spherical joints or hinges providing for customizable relative position of the interlocking elements.

According to some embodiments LEs essentially resemble a nut, threaded onto a protruding LECP, tightening the interlocking element of the inferior IBM to the respective BSM and or to the interlocking element/s of anterior IBM/s; whereby frictionally engaging the inferior IBM to the respective BSM and anterior IBM/s to each other. According to some other embodiments LEs essentially resemble a bolt threaded into IBM's cylindrical LECP indentation.

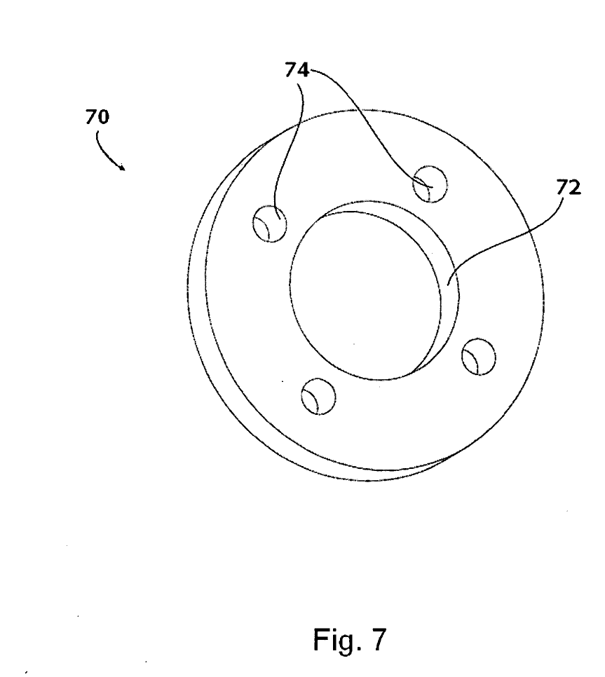

Reference is now made to FIG. 7, showing exemplary LE 70 having aperture 72 to be threaded onto protruding LECP (not shown). Holes 74 in the LE are disposed for complementary instruments.

According to the method of the present invention, after placing the distal end of BSM's bone-screwable portion against the tissue of a bone fragment, a rotational torque is applied to the IBM engaging portion or LECP, by a complementary tool implemented of such task, whereby the bone-screwable portion is screwed into the bone tissue and an anchoring point is created on the bone fragment. This procedure is repeated at least once for each bone fragment. According to the preferred embodiments the procedure is repeated at least twice for each bone fragment, whereby at least two anchoring points for each bone fragment are created. A plurality of anchoring points for each bone fragment can provide for mechanically and structurally superior properties of structural framework implemented for osteosynthesis, as will be elaborated below.

After having screwed the BSMs into the bone fragments, complementary manipulating tool or tools are operatively connected to the BSM's IBM engaging portion or LECP, for manipulating the bone fragments and bringing them into an optimal position. Once the proper positioning has been achieved, the complementary tool or tools may be further optionally held firmly, for instance by a temporal stabilization frame or some other means, stabilizing the achieved position of the bone fragments.

Subsequently, a first inferior IBM is appended to the BSMs, in such a manner that the interlocking elements thereof are applied to the IBM engaging portions of two distinct BSMs screwed into two different bone fragments. According to the preferred embodiments this procedure is repeated, with required modifications, for tandem assembly of structural framework implemented for osteosynthesis. The aforementioned required modifications include: assembling IBM in such a manner that one interlocking element thereof is applied to the IBM engaging portions of BSM, while the second interlocking element thereof is applied to the interlocking element of another IBM; assembling IBM in such a manner that the interlocking elements thereof are applied to the interlocking elements of two other different distinct IBMs.

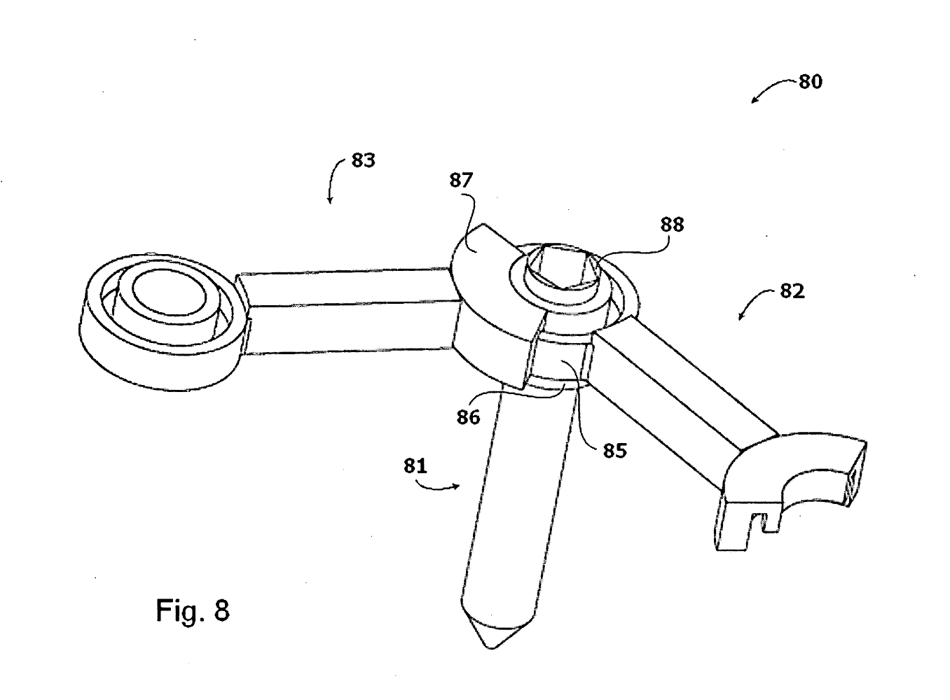

Reference is now made to FIG. 8, showing assembly 80 of exemplary BSM 81, first inferior IBM 82, and tandem assembled IBM 83. Inferior IBM 82 and tandem assembled IBM 83 are capable of pivoting around the centric axis of BSM 81 (not shown). Annular interlocking element 85 of inferior IBM 82 is applied to IBM engaging portion 86 of BSM 81. A sector of an annulus type interlocking element 87 of IBM 83 is applied to annular interlocking element 85 of inferior IBM 82 so that a ridge interacts with an opposing groove and vice versa. Subsequently LE (not shown) is threaded onto LEPC 88 and tightening assembly 80 so as to frictionally engage annular interlocking element 85 of inferior IBM 82 with IBM engaging portion 86 of BSM 81 and sector of an annulus interlocking element 87 of IBM 83 to annular interlocking element 85 of inferior IBM 82, whereby a firm module of structural framework is created.

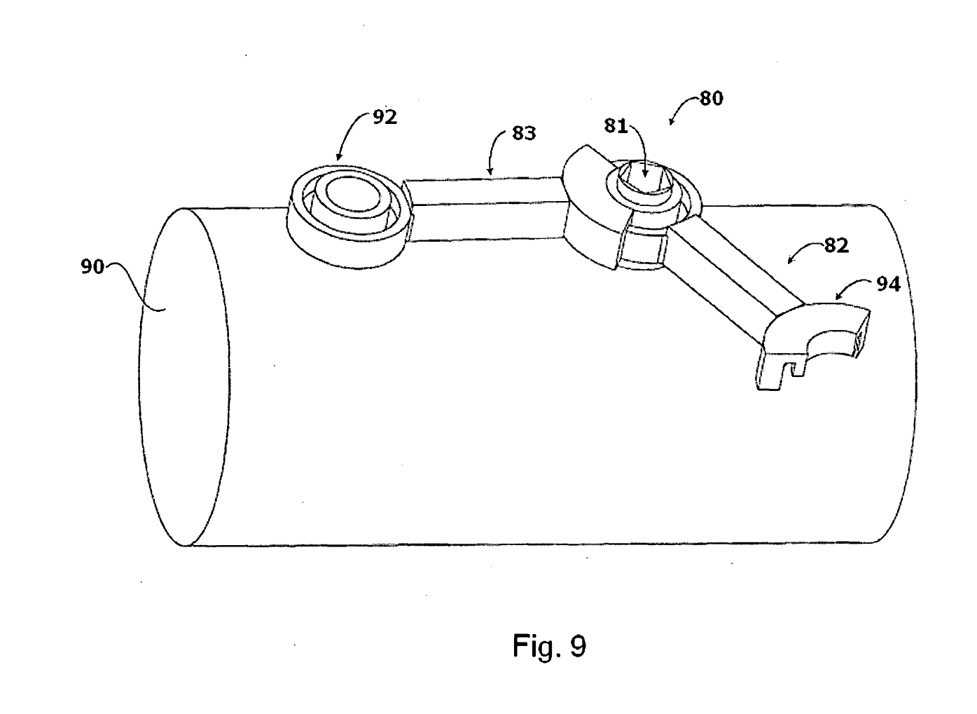

Reference is now made to FIG. 9, wherein bone-screwable portion (not shown) of BSM 81 is screwed into the tissue of bone fragment 90 and assembly 80 is thus anchored to tubular bone fragment 90, whereby one anchoring point in bone fragment 90 is created. Annular interlocking element 92 of IBM 83 is available for appending to another BSM (not shown) at a different anchoring point, and or to another IBM or IBMs (not shown); and sector of annulus interlocking element 94 of inferior IBM 82 is available for appending another IBM or IBMs (not shown); for assembling of structural framework implemented for osteosynthesis.

According to some more elaborated embodiments, concurrently with assembling IBMs to BSMs and or to other IBMs, the proper adjustments are to be made to: the IBM's telescopic constituents, conferring a desired length to the bridging elements; and or to the IBM's fixable spherical joints and or hinges, conferring a desired positional angles to the interlocking elements relatively to the corresponding bridging element.

Subsequently LEs are assembled and further tightened to the LECPs of BSMs, by matching tightening tools (these may be the same as the manipulating tools), so as to engage the interlocking elements of inferior IBMs to the IBM engaging portions of the corresponding BSMs, and or the interlocking elements of different IBMs to each other; whereby firm structural framework is created and osteosynthesis is achieved.

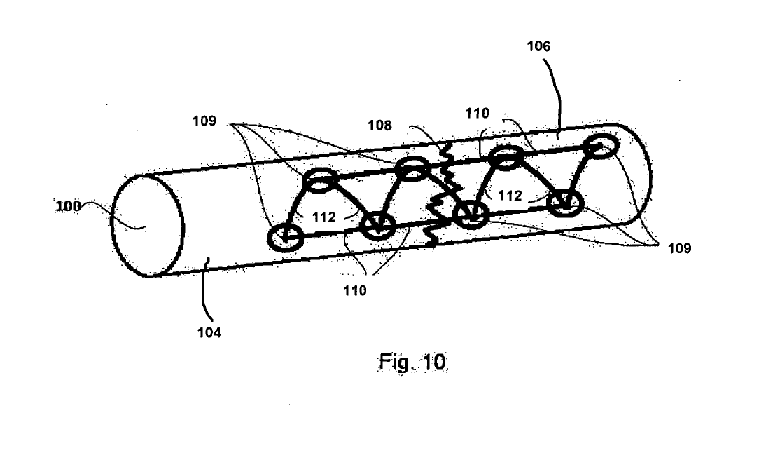

Reference is now made to FIG. 10, illustrating schematically configurational features of an assembled scaffolding in accordance with the present invention. Tubular bone 100 is fractured such that fragments 104 and 106 would have been perfectly contiguous before fracture 108 has occurred. In the treatment, fragment 104 is pierced and one or more BSMs 109 are implanted therein. After the two fragments are aligned using manipulating tools coupled to at least one BSM in the fragment, the medical crew can create the external support by interconnecting the BSMs to form an enveloping scaffolding structure around the patient's fractured bone, external to the skin. Strait IBMs 110 and arcuate IBMs 112 may be selected as required. The scaffolding obtained is rigid enough yet light weight and aerated. The scaffolding thus provided can be described as either a quasi cylindrical or forms a sector of a quasi cylinder. The resistance to bending provided by such a structure is manifested in all three dimensions. Due to the fact that the structure is curved, the potential movement of the bone fragments is restrained in all three dimensions.

The practitioner treating the bone fracture is presented with an assortment of IBMs so that a rigid connection can be created between a curved array of implanted BSMs.

If aforementioned optional stabilization of BSMs was employed earlier on, the stabilization means is removed.

It will be appreciated that the present invention is not limited by what has been particularly described and shown hereinabove and that numerous modifications, all of which fall within the scope of the present invention, exist.

Rather the scope of the invention is defined by the claims which follow:

1. A kit-of-parts to be used for assembling an external support for the fixation of orthopedic bone fractures, said kit-of-parts comprising at least one component selected from the group consisting of:
a bone-screwable member;
an interlocking bridging member (IBM), and
a locking element;
wherein said bone-screwable members are characterized by having a bone-screwable portion, an interlocking bridging member (IBM) engaging portion, and a locking element complementary portion (LECP);
wherein said interlocking bridging members are characterized by having at least two interlocking elements and a bridging element, and wherein at least one of said locking elements respectively matches at least one of said bone-screwable members, and
wherein at least one of said two interlocking elements respectively matches and is engageable with an interlocking bridging member (IBM) engaging portion of at least one of said bone-screwable members.

2. An external support assembly for the fixation of orthopedic bone fractures, said assembly comprising:
at least two bone-screwable members;
at least one interlocking bridging member, and
at least two locking elements;
wherein said bone-screwable members are screwed into the bone tissue of at least two distinct bone fragments, and said at least one interlocking bridging member is engaged with said bone-screwable members by means of said locking elements, and
wherein aforesaid members form rigid structural framework implemented for osteosynthesis.

3. The external support assembly as in claim 2, wherein said at least two bone-screwable members are a natural number of bone-screwable members selected from the group consisting of: 2, 3, 4, 5, 6, 7, 8, 9 10, 11, 12, 13, 14, 15, 16, 17, 18, 19, and 20.

4. The external support assembly as in claim 2, wherein said at least one interlocking bridging member is a natural number of interlocking bridging members greater than 3.

5. A method of assembling at least one interlocking bridging member, at least two bone-screwable members, and at least two locking elements, said process comprising the steps of:
applying said interlocking bridging member to said bone-screwable members;
applying said locking elements to said bone-screwable members, and
tightening said locking elements;
whereby said interlocking bridging member being engaged with said bone-screwable members.

6. A method of fixating and externally supporting at least two distinct bone fragments, comprising the steps of:
screwing at least two bone-screwable members, each one into a respective bone fragment, whereby creating at least one anchoring point on each respective bone fragment;
operationally connecting a manipulating tool to at least one of said bone-screwable members;
manipulating said bone-screwable member operationally connected to said manipulating tool, thereby positioning the respective bone fragment as desired;
performing the steps 1 to 3 as set forth in claim 5; thereby aforesaid members form at least a part of a rigid structural framework, and
whereby said at least two distinct bone fragments are being fixated and externally supported by said rigid structural framework.

7. The method of fixating and externally supporting at least two distinct bone fragments as in claim 6, further comprising the steps of:
stabilizing said manipulating tool after the desired position of said respective bone fragment was achieved;
removing said stabilization after said step of tightening.

8. The method of fixating and externally supporting at least two distinct bone fragments as in claim 6, wherein said at least one anchoring point on each bone fragment is a natural number of points selected from the consisting of: 2, 3, 4, 5, 6, 7, 8, 9 10, 11, 12, 13, 14, 15, 16, 17, 18, 19, and 20.

9. The method of fixating and externally supporting at least two distinct bone fragments as in claim 6, wherein said at least one interlocking bridging member is a natural number of elements selected from the consisting of: 2, 3, 4, 5, 6, 7, 8, 9 10, 11, 12, 13, 14, 15, 16, 17, 18, 19, and 20.
